(19) United States
(12) Patent Application Publication
(10) Pub. No.: US 2025/0352300 A1
(43) Pub. Date: Nov. 20, 2025

(54) METHOD AND DEVICE FOR DESIGNING DENTAL RESTORATION

(71) Applicant: AJOU UNIVERSITY INDUSTRY-ACADEMIC COOPERATION FOUNDATION, Suwon-si (KR)

(72) Inventors: Hee-Kyung KIM, Seoul (KR); Jin Woo CHOI, Suwon-si (KR); Min-Ho KYUNG, Seongnam-si (KR); Gyu Jin CHOI, Suwon-si (KR)

(21) Appl. No.: 18/850,919

(22) PCT Filed: Mar. 28, 2023
(86) PCT No.: PCT/KR2023/004121

§ 371 Date: Aug. 8, 2025

(30) Foreign Application Priority Data
Mar. 30, 2022 (KR) 10-2022-0039499

(51) Int. Cl. A61C 5/77; A61C 13/00; G16H 10/60; G16H 20/00

(52) U.S. Cl. CPC A61C 5/77; A61C 13/0004; G16H 10/60; G16H 20/00

(57) ABSTRACT

The present invention relates to a method and device for designing a dental restoration, the method comprising: processing image data related an oral cavity of a surgery subject; determining a gingival margin line of a plurality of teeth of the surgery subject on the basis of the image data; determining a dental axis of the plurality of teeth; measuring a Hausdorff distance between at least one first tooth and at least one second tooth, symmetric to the first tooth, among the plurality of teeth; and determining a degree of coincidence in the three-dimensional shape of the first tooth and the second tooth on the basis of the Hausdorff distance. Other embodiments are also possible.

### TECHNICAL FIELD

The present invention relates to a method and device for designing a dental restoration.

### BACKGROUND ART

In dentistry, auxiliary devices such as implants and dentures are applied to patients depending on the patient's dental condition. In particular, an implant refers to a substitute that can replace human tissue when the original human tissue is lost, and in dentistry, it refers to implanting an artificial tooth into the position of an actual tooth. Conventionally, before performing an implant procedure, a dentist established an implant placement plan in advance through virtual simulation by using an implant simulation program. For example, an artificial tooth that is suitable for the patient is selected, a design process is performed to virtually place the artificial tooth at the target tooth location, and the location and type of the implant structure are determined for each target tooth to be operated on.

However, when such an implant simulation is used, user operation is inconvenient, and a problem arises in which large differences in results may occur depending on the individual ability of the user. In addition, this causes a problem in that the accuracy and convenience of the implant procedure are reduced.

### DISCLOSURE

### Technical Problem

The exemplary embodiments of the present invention to solve these conventional problems provide a method and device for designing a dental restoration that can design a dental restoration to be restored at the position of a missing tooth as a mirror image of a symmetrical tooth that is symmetrical to the missing tooth.

### Technical Solution

The method for designing a dental restoration according to an exemplary embodiment of the present invention may include processing image data related an oral cavity of a surgery subject; determining a gingival margin line of a plurality of teeth of the surgery subject on the basis of the image data; determining a tooth axis of the plurality of teeth; measuring a Hausdorff distance between at least one first tooth and at least one second tooth, symmetric to the first tooth, among the plurality of teeth; and determining a degree of coincidence in the three-dimensional shapes of the first tooth and the second tooth on the basis of the Hausdorff distance.

In addition, the processing image data may include confirming a digital imaging and communications in medicine (DICOM) file based on computed tomography (CT) scan data of the oral cavity of the surgery subject; and converting the DICOM file into a first stereolithography (STL) file.

In addition, the processing image data may further include acquiring scan data for a cast model of the surgery subject; and converting the scan data into a second STL file.

In addition, the method may further include creating a final STL file by superimposing the first STL file and the second STL file.

In addition, the determining the gingival margin line may include determining the gingival margin line by setting an emergence profile for the plurality of teeth and a boundary of a crown based on the final STL file.

In addition, the determining the gingival margin line may include setting a lower boundary of the emergence profile to 3 mm below the gingival margin line.

In addition, the measuring the Hausdorff distance may include mirroring and superimposing the image of the second tooth on the image of the first tooth; and measuring the Hausdorff distance between the image of the first tooth and the image of the second tooth.

In addition, the method may further include generating and analyzing receiver operating characteristic (ROC) curve data and jitter plot data based on a threshold for the Hausdorff distance.

In addition, the generating and analyzing may include analyzing the degree of coincidence in the three-dimensional shapes of the emergence profiles for the first tooth and the second tooth and the crown on the basis of the Hausdorff distance. In addition, the determining the degree of coincidence in the three-dimensional shapes may include determining whether the degree of coincidence in the three-dimensional shapes according to the accuracy and sensitivity for the emergence profile and the crown is greater than or equal to the threshold based on the analysis results for the ROC curve data and the jitter plot data.

In addition, the threshold for the Hausdorff distance may include a value at which the specificity is 0.

Moreover, the device for designing a dental restoration according to an exemplary embodiment of the present invention may include a communicator configured to receive image data related to an oral cavity of a surgery subject from at least one acquisition device; and a processor configured to determine a gingival margin line and a tooth axis for a plurality of teeth based on the image data, and measure a Hausdorff distance between at least one first tooth and at least one second tooth, symmetric to the first tooth, among the plurality of teeth to determine a degree of coincidence in the three-dimensional shapes of the first tooth and the second tooth.

### Advantageous Effects

As described above, the method and device for designing a dental restoration according to the present invention have the effect of improving the accuracy and convenience of implant surgery by designing a dental restoration that can be restored at the position of a missing tooth as a mirror image of a symmetrical tooth that is symmetrical to the missing tooth.

### DESCRIPTION OF DRAWINGS

FIG. 1 is a diagram showing a dental restoration design system according to an exemplary embodiment of the present invention.

FIG. 2 is a diagram showing a dental restoration design device according to an exemplary embodiment of the present invention.

FIG. 3 is a flowchart for explaining a method of designing a dental restoration according to an exemplary embodiment of the present invention.

FIG. 4 is a flowchart for explaining a method of performing image processing for designing a dental restoration according to an exemplary embodiment of the present invention.

FIG. 5 is a flowchart for explaining a method of measuring the Hausdorff distance according to an exemplary embodiment of the present invention.

FIG. 6 is a diagram showing the tooth structure according to an exemplary embodiment of the present invention.

FIG. 7 is a diagram showing ROC curve data for the emergence profile boundary according to an exemplary embodiment of the present invention.

FIG. 8 is a diagram showing ROC curve data for a crown according to an exemplary embodiment of the present invention.

FIG. 9 is a diagram showing jitter plot data for the emergence profile boundary and crown according to an exemplary embodiment of the present invention.

### MODES OF THE INVENTION

Hereinafter, preferred exemplary embodiments according to the present invention will be described in detail with reference to the attached drawings. The detailed description set forth below in conjunction with the accompanying drawings is intended to illustrate exemplary embodiments of the invention and is not intended to represent the only exemplary embodiments in which the invention may be practiced. In order to clearly explain the present invention in the drawings, parts that are irrelevant to the description may be omitted, and the same reference numerals may be used for identical or similar components throughout the specification.

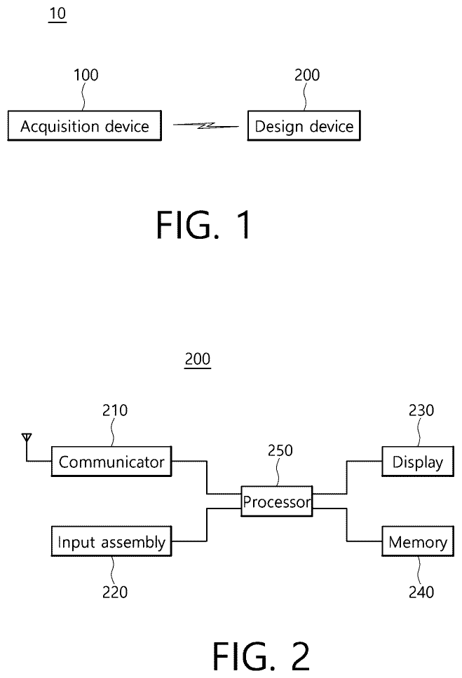

FIG. 1 is a diagram showing a dental restoration design system according to an exemplary embodiment of the present invention.

Referring to FIG. 1, the dental restoration design system 10 according to the present invention may include an acquisition device 100 and a design device 200.

The acquisition device 100 acquires image data related to the inside of the oral cavity of a surgery subject and transmits the same to the design device 200. To this end, the acquisition device 100 may perform communication, such as 5th generation mobile telecommunication (5G), long term evolution-advanced (LTE-A), long term evolution (LTE), and wideband code division multiple access (WCDMA) and Wi-Fi (wireless fidelity) communication, with the design device 200.

More specifically, the acquisition device 100 may include a CT imaging device that performs computed tomography (CT) using radiation, and a scanner device that scans a casting model that imitates the inside of the oral cavity of the surgery subject. The acquisition device 100 transmits CT imaging data about the inside of the oral cavity of the surgery subject obtained from the CT imaging device and scan data about the casting model obtained from the scanner device to the design device 200.

The design device 200 is a device that can design an implant (hereinafter, referred to as a restoration), and it may be a device such as a computer. The design device 200 processes image data including CT imaging data and scan data received from the acquisition device 100, and based on these, it determines the gingival margin line and tooth axis for a plurality of firth teeth. The design device 200 mirrors the second tooth that is symmetrical to the first tooth and measures the Hausdorff distance between the first tooth and the mirrored second tooth. The design device 200 may determine the degree of coincidence of the three-dimensional shapes of the first tooth and the second tooth on the basis of the measured Hausdorff distance. The design device 200 may design a dental restoration to be restored at the position of a missing tooth by using the symmetrical tooth of the missing tooth based on the determined degree of incidence of the three-dimensional shapes. A more detailed operation of the design device 200 will be described by using FIG. 2 below. FIG. 2 is a diagram showing a dental restoration design device according to an exemplary embodiment of the present invention.

Referring to FIG. 2, the dental restoration design device 200 according to the present invention may include a communicator 210, an input assembly 220, a display 230, a memory 240 and a processor 250.

The communicator 210 communicates with the acquisition device 100. To this end, the communicator 210 may perform communication, such as 5th generation mobile telecommunication (5G), long term evolution-advanced (LTE-A), long term evolution (LTE), wideband code division multiple access (WCDMA) and wireless fidelity (Wi-Fi).

The input assembly 220 generates input data in response to input from a user by using the design device 200. To this end, the input assembly 220 may include input assemblies such as a keyboard, mouse, keypad, dome switch, touch panel, touch keys and buttons.

The display 230 outputs output data according to the operation of the electronic device 200. To this end, the display 230 may include a display device, such as a liquid crystal display (LCD), a light emitting diode (LED) display or an organic light emitting diode (OLED) display. In addition, the display 230 may be combined with the input assembly 220 and implemented in the form of a touch screen.

The memory 240 stores operation programs of the design device 200. The memory 240 may store an image processing program for processing image data received from the acquisition device 100, an algorithm for measuring the Hausdorff distance and algorithms for generating receiver operating characteristic (ROC) curve data and jitter plot data.

The processor 250 performs image processing on image data received from the acquisition device 100 through the communicator 210. More specifically, the processor 250 transmits a request signal for acquiring computed tomography (CT) imaging data to the acquisition device 100 and confirms a digital imaging and communications in medicine (DICOM) file based on CT imaging data of the oral cavity of the surgery subject received from the acquisition device 100. The processor 250 converts the confirmed DICOM file into a first stereolithography (STL) file.

When the processor 250 completes the creation of a cast model that imitates the inside of the oral cavity of the surgery subject, it transmits a request signal for acquiring scan data for the cast model to the acquisition device 100. The processor 250 converts the scan data for the cast model received from the acquisition device 100 into a second STL file.

The processor 250 creates a new final STL file by superimposing the first STL file and the second STL file and determines the gingival margin line of the surgery subject based on the final STL file. More specifically, the processor 250 sets the emergence profile of the first and second teeth and the boundary of a crown based on the final STL file. In this case, the processor 250 may determine the gingival margin line by setting an area 3 mm below the gingival margin as the lower boundary of the emergence profile. For example, in an exemplary embodiment of the present invention, the central incisor, lateral incisor and canine located on the left side in the maxillary anterior region of the surgery subject will be described as the first teeth, and the central incisor, lateral incisor and canine located on the right side of the maxillary anterior region and are symmetrical to the first tooth, will be described as the second teeth.

The processor 250 determines the tooth axes of the first tooth and the second tooth. In this case, the processor 250 may determine the exact center of the incisal edge as the tooth axis in the case of central incisors and lateral incisors, and may determine the canine tip as the tooth axis in the case of canines.

The processor 250 separates the emergence profile area and crown area for the first and second teeth of the surgery subject from the final STL file. In this case, when separating the emergence profile areas, the processor 250 may set up to 3 mm below the gingival margin as the lower boundary of the emergence profile and separate the same into the emergence profile area.

The processor 250 may copy the image of the second tooth that is symmetrical to the first tooth in the final STL file, and mirror the copied image of the second tooth on the image of the first tooth to superimpose the images. The processor 250 measures the Hausdorff distance between the image of the first tooth and the mirrored and superimposed image of the second tooth.

The processor 250 generates and analyzes receiver operating characteristic (ROC) curve data and jitter plot data based on the measured Hausdorff distance. The processor 250 confirms the degree of coincidence in the three-dimensional shapes of the first tooth and the second tooth based on the analysis results of the ROC curve data and jitter plot data.

The processor 250 may confirm the degree of incidence in the three-dimensional shapes by checking true-positives and false-positives for the emergence profile and crown using the threshold of the Hausdorff distance. In this case, the processor 250 may set the threshold of the Hausdorff distance to a value at which the specificity is 0.

More specifically, the processor 250 may check the true positive rate, false positive rate, pair matching accuracy and sensitivity for the emergence profiles of the first tooth and the second tooth, and confirm the degree of coincidence in the three-dimensional shapes by confirming the true positive rate, false positive rate, pair matching accuracy and sensitivity for the crowns of the first tooth and the second tooth. If the confirmed 3D shape coincidence information is greater than or equal to the threshold, the processor 250 may determine that a restoration to be restored at the location of a missing tooth may be designed with a symmetrical tooth that is symmetrical to the missing tooth.

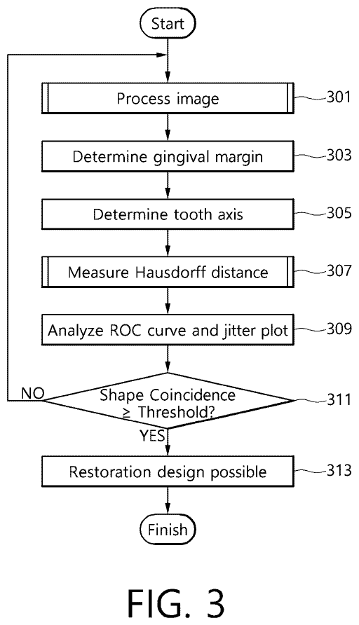

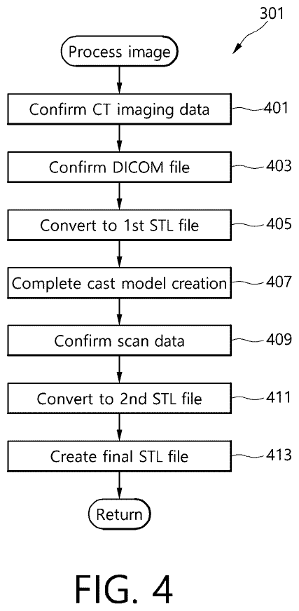

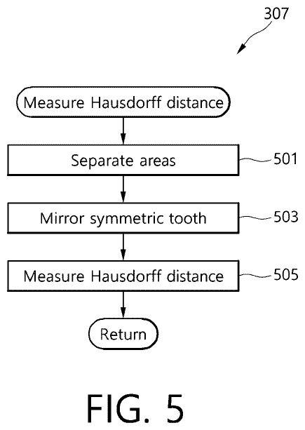

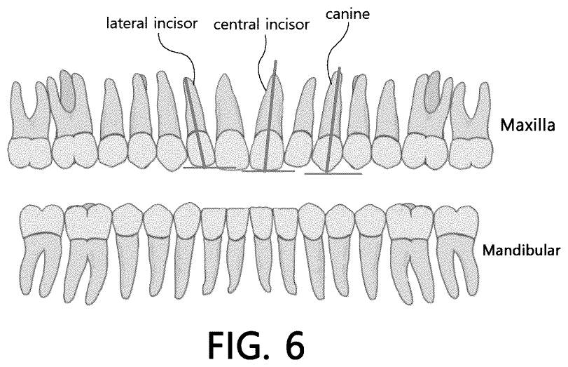

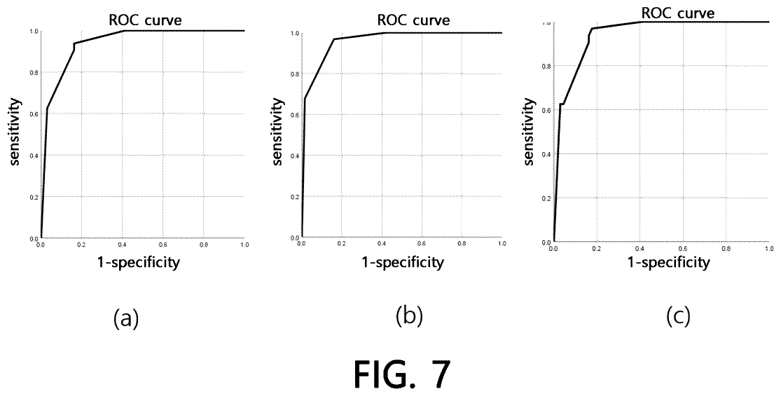

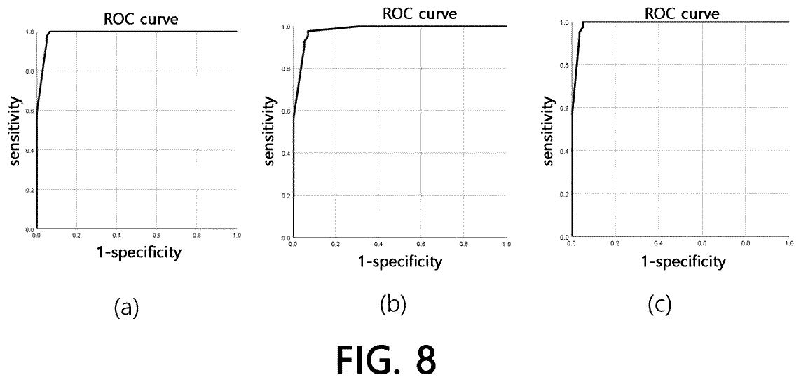

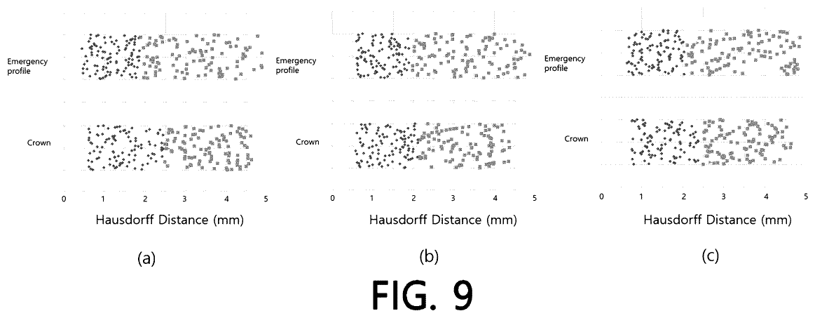

FIG. 3 is a flowchart for explaining a method of designing a dental restoration according to an exemplary embodiment of the present invention. FIG. 4 is a flowchart for explaining a method of performing image processing for designing a dental restoration according to an exemplary embodiment of the present invention. FIG. 5 is a flowchart for explaining a method of measuring the Hausdorff distance according to an exemplary embodiment of the present invention. FIG. 6 is a diagram showing the tooth structure according to an exemplary embodiment of the present invention. FIG. 7 is a diagram showing ROC curve data for the emergence profile boundary according to an exemplary embodiment of the present invention. FIG. 8 is a diagram showing ROC curve data for a crown according to an exemplary embodiment of the present invention. FIG. 9 is a diagram showing jitter plot data for the emergence profile boundary and crown according to an exemplary embodiment of the present invention.

Referring to FIGS. 3 to 9, in step 301, the processor 250 performs image processing on image data received from the acquisition device 100 through the communicator 210. In this case, a specific method for image processing will be described by using FIG. 4 below. Referring to FIG. 4, in step 401, the processor 250 transmits a request signal for the acquisition of computed tomography (CT) imaging data to the acquisition device 100, and checks CT imaging data for the inside of the oral cavity of the surgery subject received from the acquisition device 100. In step 403, the processor 250 checks a digital imaging and communications in medicine (DICOM) file based on the CT imaging data and performs step 405. In step 405, the processor 250 converts the DICOM file into a first stereolithography (STL) file.

Subsequently, in step 407, the processor 250 performs step 409 when the creation of a cast model that imitates the inside of the oral cavity of the surgery subject is completed. In step 409, the processor 250 transmits a request signal for acquiring scan data for the cast model to the acquisition device 100 and acquires scan data for the cast model received from the acquisition device 100. In step 411, the processor 250 converts the scan data into a second STL file.

In step 413, the processor 250 creates a new final STL file by superimposing the first STL file converted in step 405 and the second STL file converted in step 411 and returns to step 303 in FIG. 3. In this way, the present invention has the effect of generating a more accurate final STL file for the inside of the oral cavity of the surgery subject by superimposing the first STL file and the second STL file. Moreover, in an exemplary embodiment of the present invention, the order of checking the CT imaging data and then checking the scan data for the cast model is described, but the order is not necessarily limited thereto, and the CT imaging data may be checked after checking the scan data.

In step 303, the processor 250 determines the gingival margin line of the surgery subject based on the final STL file. More specifically, the processor 250 sets the emergences profiles of the first and second teeth and the boundary of the crown based on the final STL file. In this case, the processor 250 may determine the gingival margin line by setting the area 3 mm below the gingival margin as the lower boundary of the emergence profile. For example, the processor 250 may determine the gingival margin line for each of the central incisor, lateral incisor and canine located on the left side in the maxillary incisors, which are the first teeth, and the central incisor, lateral incisor and canine located on the right side in the maxillary incisors, which are the second teeth.

Next, in step 305, the processor 250 determines the tooth axis of the surgery subject. In this case, as shown in FIG. 6, the processor 250 may determine the exact center of the incisal edge as the tooth axis in the case of central incisors and lateral incisors and may determine the canine tip as the tooth axis in the case of canines.

In step 307, the processor 250 measures the Hausdorff distance. This will be explained in more detail by using FIG. 5 below. Referring to FIG. 5, in step 501, the processor 250 separates the emergence profile area and crown area for the first and second teeth from the final STL file. In this case, when separating the emergence profile area, the processor 250 may set up to 3 mm below the gingival margin as the lower boundary of the emergence profile and separate the same into the emergence profile area.

Next, the processor 250 performs step 503. In step 503, the processor 250 copies the image of the second tooth that is symmetrical to the first tooth in the final STL file. The processor 250 may mirror the copied image of the second tooth with the image of the first tooth to superimpose the images. For example, the processor 250 may copy the image of the tooth that is symmetrical to the central incisor located on the left side in the maxillary anterior region of the surgery subject, that is, the image of the central incisor located on the right side in the maxillary anterior region, mirror the image of the central incisor located on the left side, and superimpose the same. In step 505, the processor 250 measures the Hausdorff distance between the image of the first tooth and the image of the second tooth that is mirrored and superimposed with the image of the first tooth, and the processor 250 returns to step 309 of FIG. 3.

In step 309, the processor 250 generates and analyzes receiver operating characteristic (ROC) curve data and jitter plot data based on the measured Hausdorff distance. In this case, the ROC curve refers to a curve showing changes in the true positive rate (TPR) and the false positive rate (FPR), and it is a graph with TPR and FPR on the y-axis and x-axis, respectively. In the present invention, the rate (sensitivity) of correctly predicting 1 when the TPR is 1 means the rate of predicting that a pair of symmetrical teeth, for example, a first tooth and a second tooth, match. In addition, the rate of incorrectly predicting 1 when FPR is 0 (1-specificity) refers to the rate of predicting that a pair of symmetrical teeth do not match as coincidence. Moreover, sensitivity and 1-specificity are inversely proportional. In addition, jitter plot refers to one of the visualization techniques that shows the relationship between variables.

Referring to FIGS. 7 and 8, the ROC curve data according to the present invention marks the sensitivity and 1-specificity for the image of the second tooth mirrored in the image of the first tooth based on the threshold of the Hausdorff distance, and it is data that has analyzed the emergence profile and 3D shape coincidence information of the crown.

(a) of FIG. 7 shows ROC curve data for the emergence profile boundary of the central incisors, (b) of FIG. 7 shows ROC curve data for the emergence profile boundary of the lateral incisors, and (c) of FIG. 7 shows ROC curve data for the emergence profile boundary of the canines. Moreover, (a) of FIG. 8 shows ROC curve data for the crown of the central incisor, (b) of FIG. 8 shows ROC curve data for the crown of the lateral incisor, and (c) of FIG. 8 shows ROC curve data for the crown of the canine. In the ROC curve data, the closer the ROC curve is in the TPR direction, the more accurate the result is. Therefore, when the image of the second tooth is mirrored on the image of the first tooth as shown in FIGS. 7 and 8, it can be confirmed that the sensitivity of both the emergence profile boundary and the crown is close to 1, and 1-specificity is close to 0. Therefore, when the image of the second tooth is mirrored on the image of the first tooth, it can be predicted that the left and right tooth pairs match.

(a) of FIG. 9 shows a distribution chart of the emergence profile boundary of the central incisor and the crown shown based on the threshold of the Hausdorff distance, (b) of FIG. 9 shows a distribution chart of the emergence profile boundary of the lateral incisor and the crown, and (c) of FIG. 9 shows a distribution chart of the emergence profile boundary of the canine and the crown. In this case, the blue distribution is a distribution that predicted the actual true positive as a true positive, the orange distribution is a distribution that predicted the actual true negative as a true negative, and the gray distribution is a distribution that predicted the true positive as a false negative. Looking at FIG. 9, it was judged that the teeth that were true positive and symmetrical up to a Hausdorff distance of about 2 mm were actually similar, and since there was almost no gray distribution, it can be confirmed that there are almost no false negatives.

Next, in step 311, the processor 250 determines whether the degree of incidence in the three-dimensional shapes between the image of the first tooth and the image of the second tooth is greater than or equal to a threshold according to the analysis result of step 309. The processor 250 may check the degree of coincidence in the three-dimensional shapes by checking true-positives and false-positives for the emergence profile and crown using the threshold of the Hausdorff distance. In this case, the processor 250 may set the threshold of the Hausdorff distance to a value at which the specificity is 0.

As a result of the confirmation in step 311, if the degree of coincidence in the three-dimensional shapes is greater than or equal to the threshold, the processor 250 performs step 313. In step 313, the processor 250 may determine that a restoration to be restored at the position of a missing tooth may be designed with a symmetrical tooth that is symmetrical to the missing tooth, because the degree of coincidence in the three-dimensional shapes of the first tooth and the second tooth is greater than or equal to the threshold.

The three-dimensional shape coincidence results confirmed by mirroring the image of the second tooth on the image of the first tooth as in the present invention are as follows. In the case of emergence profiles, the true positive rates for central incisors, lateral incisors and canines are 0.99, 1.00, and 0.98, and the false positive rates are 0.01, 0.00 and 0.02, respectively. In addition, the pair matching accuracy is 0.99 or more, and the sensitivity is 0.98 or more. In the case of crowns, the true positive rates for central incisors, lateral incisors and canines are 1.00, 0.98, and 0.98, and the false positive rates are 0.00, 0.02 and 0.02, respectively. In addition, the pair matching accuracy is 0.99 is more, and the sensitivity is 0.98 or more.

As such, the present invention may create a restoration including a crown, fixture and the like by using a mirrored image of a symmetrical tooth that is symmetrical to a missing tooth when creating a restoration for the restoration of the missing tooth of the surgery subject in the maxillary anterior region. Through this, the present invention has the effect of improving the accuracy, convenience and aesthetics of the implant procedure. Moreover, in an exemplary embodiment of the present invention, the maxillary anterior teeth of the surgery subject are described as an example, but this is only for the convenience of explanation, and the present invention is not necessarily limited thereto.

The exemplary embodiments of the present invention disclosed in the present specification and drawings are merely provided as specific examples to easily explain the technical content of the present invention and to facilitate understanding of the present invention and are not intended to limit the scope of the present invention. Therefore, the scope of the present invention should be construed as including all changes or modified forms derived based on the technical idea of the present invention in addition to the exemplary embodiments disclosed herein.

1. A method for designing a dental restoration, the method comprising:
processing image data related to an oral cavity of a surgery subject;
determining a gingival margin line of a plurality of teeth of the surgery subject on the basis of the image data;
determining a tooth axis of the plurality of teeth;
measuring a Hausdorff distance between at least one first tooth and at least one second tooth, symmetric to the first tooth, among the plurality of teeth; and
determining a degree of coincidence in the three-dimensional shapes of the first tooth and the second tooth on the basis of the Hausdorff distance.

2. The method of claim 1, wherein the processing image data comprises:
confirming a digital imaging and communications in medicine (DICOM) file based on computed tomography (CT) scan data of the oral cavity of the surgery subject; and
converting the DICOM file into a first stereolithography (STL) file.

3. The method of claim 2, wherein the processing image data further comprises:
acquiring scan data for a cast model of the surgery subject; and
converting the scan data into a second STL file.

4. The method of claim 3, further comprising:
creating a final STL file by superimposing the first STL file and the second STL file.

5. The method of claim 4, wherein the determining the gingival margin line comprises:
determining the gingival margin line by setting an emergence profile for the plurality of teeth and a boundary of a crown based on the final STL file.

6. The method of claim 5, wherein the determining the gingival margin line comprises:
setting a lower boundary of the emergence profile to 3 mm below the gingival margin line.

7. The method of claim 5, wherein the measuring the Hausdorff distance comprises:
mirroring and superimposing the image of the second tooth on the image of the first tooth; and
measuring the Hausdorff distance between the image of the first tooth and the image of the second tooth.

8. The method of claim 7, further comprising:
generating and analyzing receiver operating characteristic (ROC) curve data and jitter plot data based on a threshold for the Hausdorff distance.

9. The method of claim 8, wherein the generating and analyzing comprises:
analyzing the degree of coincidence in the three-dimensional shapes of the emergence profiles for the first tooth and the second tooth and the crown on the basis of the Hausdorff distance.

10. The method of claim 9, wherein the determining the degree of coincidence in the three-dimensional shapes comprises:
determining whether the degree of coincidence in the three-dimensional shapes according to the accuracy and sensitivity for the emergence profile and the crown is greater than or equal to the threshold based on the analysis results for the ROC curve data and the jitter plot data.

11. The method of claim 8, wherein the threshold for the Hausdorff distance includes a value at which the specificity is 0.

12. A device for designing a dental restoration, the device comprising:
a communicator configured to receive image data related to an oral cavity of a surgery subject from at least one acquisition device; and
a processor configured to:
determine a gingival margin line and a tooth axis for a plurality of teeth based on the image data,
measure a Hausdorff distance between at least one first tooth and at least one second tooth, symmetric to the first tooth, among the plurality of teeth, and
determine a degree of coincidence in the three-dimensional shapes of the first tooth and the second tooth.
